(19) United States
(12) Patent Application Publication
(10) Pub. No.: US 2025/0226103 A1
(43) Pub. Date: Jul. 10, 2025

(54) SYSTEMS AND METHODS FOR PREDICTING TRAVEL-RELATED STRESS

(71) Applicant: Rebook, Inc., Boston, MA (US)

(72) Inventors: Timothy KASBE, Hauraki (NZ); Byron REEVES, Boston, MA (US)

(21) Appl. No.: 19/095,654

(22) Filed: Mar. 31, 2025

Related U.S. Application Data
(63) Continuation of application No. PCT/US2023/075337, filed on Sep. 28, 2023.
(60) Provisional application No. 63/411,684, filed on Sep. 30, 2022.

(51) Int. Cl. G16H 50/20; G16H 20/70; G16H 50/70

(52) U.S. Cl. CPC G16H 50/20; G16H 20/70; G16H 50/70

(57) ABSTRACT

Techniques for generating training data for a model configured to predict stress are provided, including receiving, for each person of a plurality of people, data associated with a stress level of the person, the data including data having different data types, the different data types including a first type and a second type, categorizing each person of the plurality of people as belonging to a stress group of a plurality of stress groups, generating, using a variational autoencoder configured to use a first distribution to encode and sample data of the first type and a second distribution to encode and sample data of the second type, augmented data for each group of the plurality of stress groups, the augmented data including data including data having the first type and data have the second type, and outputting the augmented data as training data for training a model to predict stress.

### CROSS REFERENCE TO RELATED APPLICATIONS

This application is a Continuation of International Patent Application No. PCT/US2023/075337, filed Sep. 28, 2023, titled “Systems and Methods for Predicting Travel-Related Stress,” which claims priority to U.S. Provisional Patent Application No. 63/411,684, filed Sep. 30, 2022 and titled “Systems and Methods for Predicting Travel-Related Stress,” the contents of each of which are hereby incorporated by reference in their entireties, for all purposes.

### TECHNICAL FIELD

This disclosure relates generally to techniques for predicting travel-related stress.

### BACKGROUND

Whether it is traveling to a hospital emergency room across town, traveling on vacation with a family, or business travel to an important meeting, travel can induce various levels of stress in individuals. Travel-related applications are designed to facilitate the process of booking and/or cancelling travel accommodations (e.g., airfare, hotels, rental cars, etc.) for customers, but do not typically take into account the stress level of the customer during that process.

### SUMMARY

Disclosed are automated systems and methods for predicting a level of travel-related stress that a person is experiencing or is likely to experience based on a collection of inputs from various sources. In some embodiments, a communicative context of a travel-based application (e.g., via a user interface (UI) of a mobile application) may be configured based on the predicted stress level. In some implementations, such predictions may be made by evaluating the various inputs using one or more trained machine learning (ML) models. In some implementations, a variational autoencoder may be used to generate augmented data used to train the one or more ML models. The variational autoencoder may use different distributions to encode and sample feature values depending on a type of data provided as input to the variational autoencoder. Feature value sets output from the variational autoencoder for different data types may be combined to provide a multi-type (e.g., continuous, categorical, binary) training data set for users having different stress levels, which may be used to train a predictive model of travel-related stress. In some embodiments, the training data set may also include data captured in multiple time-domains (e.g., data related to moment-to-moment stress in relation to automatic arousal and other fast changing physical manifestations of stress, stress experienced as relatively enduring states over hours, days or weeks, etc.).

In some embodiments, a method of generating training data for a model configured to predict stress is provided. The method includes receiving, for each person of a plurality of people, data associated with a stress level of the person, the data including data having different data types, the different data types including a first type and a second type, categorizing each person of the plurality of people as belonging to a stress group of a plurality of stress groups, generating, using a variational autoencoder configured to use a first distribution to encode and sample data of the first type and a second distribution to encode and sample data of the second type, augmented data for each group of the plurality of stress groups, the augmented data including data including data having the first type and data have the second type, and outputting the augmented data as training data for training a model to predict stress.

In one aspect, the first type is categorical and the second type is continuous. In another aspect, the first distribution is a Poisson distribution and the second distribution is a Gaussian distribution. In another aspect, the different data types further include a third type, the variational autoencoder is configured to use a third distribution to encode and sample data of the third type, and the augmented data includes data having the third type. In another aspect, the first type is categorical, the second type is continuous and the third type is binary. In another aspect, the first distribution is a Poisson distribution, the second distribution is a Gaussian distribution, and the third distribution is a Bernoulli distribution. In another aspect, the plurality of stress groups include a low stress group and a high stress group.

In another aspect, the plurality of stress groups further include a moderate stress group. In another aspect, generating the augmented data includes providing, as input to the variational autoencoder, data of the first type for a first stress group of the plurality of stress groups, encoding, by an encoder of the variational autoencoder, features of data of the first type in a latent space according to the first distribution, sampling first feature values for data of the first type encoded in the latent space, decoding, by a decoder of the variational autoencoder, the first feature values to generate first output data having the first type, providing, as input to the variational autoencoder, data of the second type for the first stress group of the plurality of stress groups, encoding, by the encoder of the variational autoencoder, features of data of the second type in the latent space according to the second distribution, sampling second feature values for data of the second type encoded in the latent space, decoding, by the decoder of the variational autoencoder, the second feature values to generate second output data having the second type, and combining the first output data and the second output data to generate the augmented data for the first stress group.

In some embodiments, a method of training a predictive model to predict stress is provided. The method includes receiving a training data set, the training data set including labeled data having different data types including a first type and a second type, each instance of the labeled data in the training data set having values associated with a label indicating a stress level, training, using the training data set, a predictive model relating the values in the instances of the labeled data to the labels indicating a stress level, and configuring at least one computer system to predict the stress of a user using the trained predictive model and data values associated with the user.

In one aspect, the predictive model includes at least one neural network. In another aspect, the at least one neural network includes at least one multilayered perceptron including an input layer, an output layer and one or more hidden layers. In another aspect, the output layer is coupled to a random forest classifier.

In some embodiments, a method of configuring a communicative context between a user and a user interface of a travel-related application is provided. The method includes receiving a plurality of data values associated with the user, the plurality of data values having at least a first data type and a second data type, determining a stress level of the user based, at least in part, on the plurality of data values associated with the user and a predictive model trained to predict a stress level of user based on data having values of the first data type and the second data type, and configuring a communicative context of a travel-related application based, at least in part, on the determined stress level of the user.

In one aspect, determining the stress level of the user comprises providing at least some of the plurality of data values as input to the predictive model, wherein an output of the predictive model is one stress level of a plurality of stress levels. In another aspect, the plurality of stress levels includes a low stress level and a high stress level. In another aspect, the plurality of stress levels further includes a moderate stress level. In another aspect, configuring a communicative context of a travel-related application comprises selecting a style of communication with the user via the travel-related application based on the determined stress level of the user. In another aspect, configuring a communicative context of a travel-related application comprises selecting content to provide to the user via the travel-related application based on the determined stress level of the user. In another aspect, configuring a communicative context of a travel-related application comprises modifying a communication protocol of the travel-related application based on the determined stress level of the user.

In some embodiments, a non-transitory computer-readable medium encoded with a plurality of instructions is provided. The plurality of instructions when executed by at least one computer processor perform a method according to any of methods described herein. In some embodiments, a computer system is provided. The computer system includes at least one computer processor at least one non-transitory computer-readable medium encoded with a plurality of instructions. The plurality of instructions when executed by the at least one computer processor performs a method according to any of the methods described herein.

The advantages of the invention, together with further advantages, may be better understood by referring to the following description taken in conjunction with the accompanying drawings.

### DESCRIPTION OF DRAWINGS

FIG. 1 is a flowchart of a process for configuring a communicative context of a travel-based application based on user stress, in accordance with some embodiments.

FIG. 2 is a flowchart of a process for generating augmented data having different types, in accordance with some embodiments.

FIG. 3 is a flowchart of a process for training a predictive model, in accordance with some embodiments.

FIG. 4 schematically illustrates a variational autoencoder architecture that may be used in accordance with some embodiments to generate augmented data used for training a predictive model, in accordance with some embodiments.

FIG. 5 schematically illustrates an architecture for a predictive model that may be used to predict travel-related stress, in accordance with some embodiments.

FIG. 6 is a block diagram of a process for training and using a predictive model to predict travel-related stress, in accordance with some embodiments.

FIG. 7 schematically illustrates examples of predictive variables that may be used to predict travel-related stress, in accordance with some embodiments.

### DETAILED DESCRIPTION

Traveling helps to relieve stress, but for some people, it can induce travel-related stress, a feeling of mental strain, and pressure relating to travel. For instance, travel-related stress can lead to a bad vacation experience. Getting on a plane is often one of the most stressful experiences for international and business travelers. There are a variety of factors that can affect the stress levels of people traveling. The most common air travel stressors include the long check-in lines, large airports with complicated layouts, reservation problems, baggage fees, security issues, flight delays, cancellations, and overbookings. Some embodiments of the technology described herein relate to techniques for training and/or using a predictive model of travel-related stress. The predictive model may be used to, for example, predict a moment-to-moment level of stress of an individual and change, based on the predicted stress level, how a travel-related application (e.g., installed on a user's mobile device) is configured to communicate with the individual. For example, when the user's level of stress is relatively low, the travel-related application may communicate with the user in a light-hearted manner (e.g., by using humor or otherwise), whereas when the user's level of stress is relatively high, the travel-related application may communicate with the user using a soothing tone, by providing less information, or by using some other communicative technique that aligns with, rather than exacerbates, the user's current stress level. For instance, how content is provided to the user via the travel-related application and/or what content is provided to the user via the travel-related application may be adjusted based on a current stress level of the user output from the predictive model.

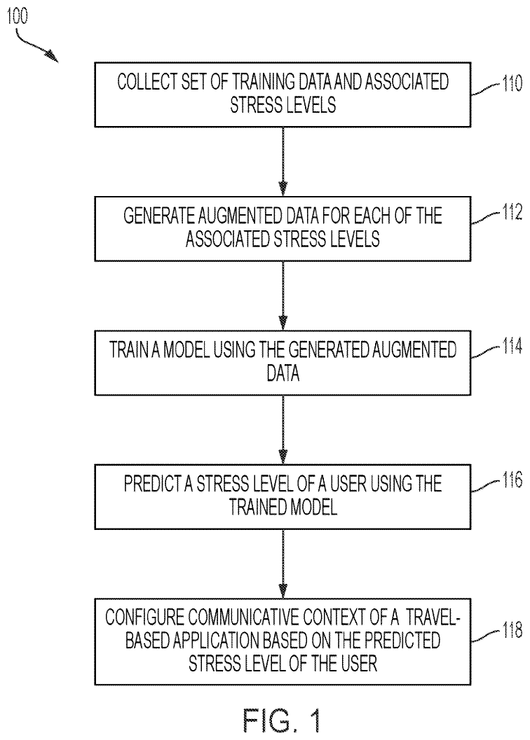

FIG. 1 is a flowchart of a process 100 for configuring a communicative context of a travel-based application using a predictive model of stress in accordance with some embodiments. In act 110, a set of training data is collected and training data is labeled with associated stress levels (e.g., low stress, moderate stress, high stress). The training data, examples of which are described herein, may include data provided from various sources and may have different types (e.g., categorical data, binary data, continuous data). Additionally, the training data may include data in different time domains (e.g., moment-by-moment changes, hour or daily changes, etc.). In some embodiments, the set of features collected as training data includes at least one travel-specific stressor (e.g., number of flight cancellations at an airport) and at least one physical measurement (e.g., typing fast).

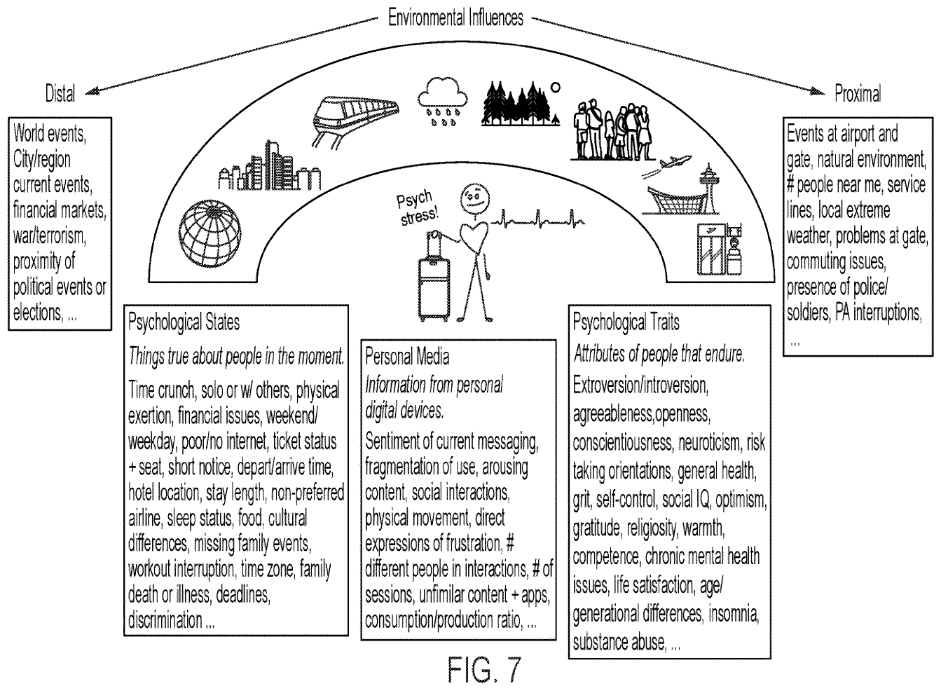

The training data may include values of predictor variables associated with different categories. FIG. 7 schematically illustrates five different categories of predictor variables that may be used as training data to train a predictive model in accordance with some embodiments. As shown in FIG. 7, the categories include proximate environment influences, distal environment influences, psychological traits of the user (e.g., attributes of traveler psychology that are relatively fixed), psychological states of the user (e.g., attributes of traveler psychology important in the moment), and personal media use (e.g., psychological information from mobile devices (e.g., URL and app logs available from the operating system of the user's mobile device)).

In some embodiments, the values for one or more of the predictor variables may be sourced from existing public databases (e.g., weather, political unrest) or from direct assessments of user psychology gathered during travel-application based onboarding questionnaires and/or periodically during travel (e.g., personality characteristics, self-reports of current psychological states). The information captured during travel may include direct questionnaires (e.g., via Ecological Momentary Assessment (EMA) questionnaires), direct observation of stress reactions (e.g., data shared via smartwatches and/or personal devices like Fitbit stress sensors), or may be inferred from the ways in which people use personal technology (e.g., typing speed, natural language sentiment analysis of language composed and received).

Non-limiting examples of proximal environment features include:
Airport size (e.g., classified based on the size in acres)
Overall occupancy of the airport (e.g., parking, total use metrics)
Local transportation issues (e.g., on-time bus/subway/train performance)
Crowd and line sizes at or near airport gate(s)
Local weather conditions (e.g., local extreme weather)
Delays at the airport
Cancellations or delays at the airport
Presence of security and police in the airport
Dining facilities at the airport

Non-limiting examples of distal environment features include:
Composite index of world conflict (e.g., wars, revolutions, political upheaval)
Financial market uncertainties and distress
Geo-political conditions in the region
National political or civic disturbances
National or international weather events
National or regional flight status and cancellation trends

Non-limiting examples of psychological trait features include:
Personality (e.g., from personality psychology literature about the “big five” traits of extroversion/introversion, agreeableness, openness, conscientiousness, neuroticism)
Risk taking orientations (e.g., with respect to being on time)
General health issues (e.g., composite indices from literature using standardized health questionnaires)
Life satisfaction surveys (e.g., from literature)

Non-limiting examples of psychological state features include:
Time urgency
Level of physical exertion
Weekday or weekend travel
Current ticket status and seat assignment
Length of trip in days/hours
Sleep and food status
Time zone changes
Family status (e.g., death, illness)
Known business or family deadlines
Hotel arrangements (e.g., quality, distance from airport and destination)

In some embodiments, personal media use metrics may be used to discern the emotional characteristics of a user's information context that may affect their stress level.

Non-limiting examples of personal medial use features include:
Sentiment analysis of current messaging (e.g., natural language processing (NLP) assessment via language tracking)
Fragmentation of digital information (e.g., speed of task switching, number and length of device sessions)
Physical movement (e.g., steps, global positioning sensor (GPS) location changes, accelerometer speed metrics)
Use of new or unfamiliar apps and content
Information production-consumption metrics (e.g., ratio of incoming to outgoing information)

In some embodiments, a stress level for a user may be determined based, at least in part, on answers to questions provided to the user (e.g., via the travel-related application) at various times. In this way, data associated with a particular user (e.g., via their mobile device) acquired as part of a training data set may be associated with a stress level label, and the labeled data may be used to train a predictive model of travel-related stress. Non-limiting examples of questions that may be provided to the user include:
How often have you been upset because of something that happened unexpectedly?
How often have you felt that you were unable to control the important things about your travel?
How often have you felt nervous and stressed?
How often have you felt confident about your ability to handle your journey plans?
How often have you felt that things were going your way?
How often have you found that you could not cope with everything you had to do?
How often have you been able to control irritations in your daily life?
How often have you felt that you were on top of things?
How often have you been angered because of things that were outside your control?
How often have you felt difficulties were piling up so high that you could not overcome them?

In some embodiments, the questions may be presented to the user 24 hours before scheduled travel, 12 hours before the scheduled travel, and after check-in for the scheduled travel. It should be appreciated, however, that the questions may be presented to the user at any other time and/or interval. The user may be asked to answer the questions considering the events that took place in the last two days. In some embodiments, the answers to one or more of the questions may be rated on a scale of 1 to 5, then using the standard Perceived Stress Scale (PSS), a stress score may be calculated and the user may be classified as having a particular stress level (e.g., Low Stress, Moderate Stress, High Stress, etc.).

In some embodiments, the training data may include data received from a travel-related application (e.g., installed on a user's mobile device). Non-limiting examples of data received from a travel-related application include:

| | Collected four times a day | |
|---|---|---|
| Daily Mean | (0-6, 6-12, 12-18, 18-24 hr) | Missed? |
| No of footsteps | No of footsteps | Yes/No |
| Duration on foot | Duration on foot | Yes/No |
| Duration Still | Duration Still | Yes/No |
| Duration - Unknown | Duration - Unknown | Yes/No |
| Duration of Conversations | Duration of Conversations | Yes/No |
| Number of conversations | Number of conversations | Yes/No |
| No of Calls | No of Calls | Yes/No |
| Distance Travelled | Distance Travelled | Yes/No |
| Phone Unlock Duration | Phone Unlock Duration | Yes/No |
| Number times phone unlocked | Number times phone unlocked | Yes/No |
| Sleep Start time | | Yes/No |
| Sleep End time | | Yes/No |
| Sleep time | | Yes/No |
| Charging time | | |

In some embodiments, the data in the above table may be acquired two days prior to the day of scheduled travel and on the day before the scheduled travel. It should be appreciated, however, that the data may be acquired from the user at any other time and/or interval.

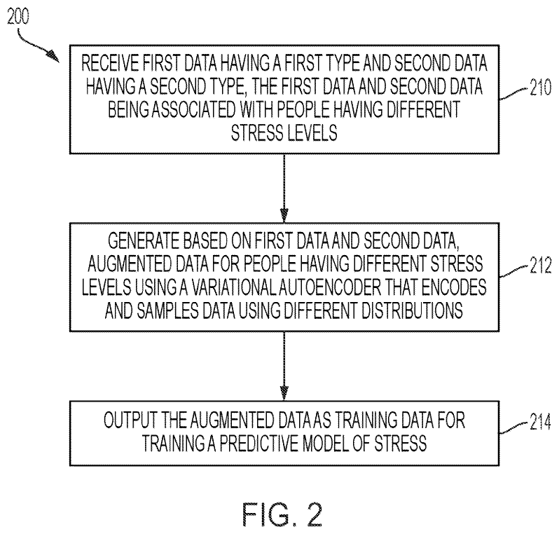

The inventors have recognized and appreciated that acquiring large amounts of travel-related stress training data from a diverse group of individuals is challenging. Accordingly, some embodiments relate to a technique for generating synthetic or augmented data based on a limited set of labeled training data. Returning to process 100, after obtaining a set of labeled training data in act 110, process 100 proceeds to act 112, where augmented data is generated for each of a plurality of stress levels. For instance, the plurality of stress levels may include three stress levels low, moderate, and high stress as determined, for example, based on answers to questions on a questionnaire and a perceived stress scale (PSS) as described above. Augmented data may be generated for each of the plurality of stress levels, such that the augmented data more accurately characterizes a wider population of individuals, which may not be represented explicitly in the collected training data. Techniques for generating augmented data in accordance with some embodiments are described in more detail below with regard to process 200 shown in FIG. 2.

Process 110 then proceeds to act 114, where the generated augmented data and/or the collected training data are used to train a predictive model for predicting stress of a user. In some embodiments, the predictive model is implemented using at least one neural network, and training the at least one neural network includes determining one or more weights for nodes in the network that enable the neural network to discriminate between users having different stress levels. Process 110 then proceeds to act 116, where the trained model is used to predict the stress level of a user. For instance, the user may be traveling to an airport to catch their flight and data (e.g., a new feature set corresponding to some or all of the data types described herein) may be acquired and provided as input to the trained predictive model. If the input data correlates highly with one of the plurality of stress levels on which the predictive model was trained, the user may be determined as having that stress level at the current time. Process 100 then proceeds to act 118, where a communicative context of a travel-based application is configured based, at least in part, on the predicted stress level of the user output from the predictive model. For instance, how the application is operating may be changed to provide information in a softer tone if the user has a high stress level. Some examples of configuring a communicative context of a travel-based application in accordance with some embodiments of the present disclosure are provided below:
Change of interface language to match the stress level of users (e.g., politeness, empathy, level of aggressiveness in resolution of travel changes and delays)
Selection of alternative travel arrangements (e.g., considerations of requirements to move quickly, urgency of travel changes)
Pictorial, audio and graphic accompaniments in interface to match and/or counteract stress levels and/or the need to move travelers to new locations quickly
Use of different personas (e.g., chatbots or avatars) in automated dialogue systems that communicate with users (to match personality of users)
Suggestions and offers for airport amenities during travel delays and changes in arrangements (e.g., food and beverage offers near customer gates, retail offers/suggestions for airport shopping, movies, health and spa, religious services)

FIG. 2 illustrates a process 200 for generating augmented data that may be used to train a predictive model of travel-related stress in accordance with some embodiments. In act 210, first data having a first type (e.g., categorical data) and second data having a second type (e.g., continuous data) may be received. For example, as described above, data (e.g., training data) from various sources (both travel-related and not travel-related) may be collected. Although data having only two types is shown in the example of FIG. 2, it should be appreciated that data having any number of types greater than two may also be used. For instance, in some implementations, categorical data, continuous data, and binary data may be collected and used for generating an augmented data set used for training a predictive model. The first data and the second data (and possibly third data, fourth data, etc.) may be associated with a person having a particular stress level determined, for example, using the perceived stress scale (PSS) described above. Accordingly, the first data and second data received in act 210 may be associated with a population of people having different stress levels. In one implementation, data having three different types (categorical, binary, and continuous data) may be collected for people having one of three stress levels (low, moderate, and high stress). In such an implementation, subsets of the collected data (e.g., categorical data for low stress people, binary data for high stress people, etc.) may be formed and used to generate augmented data using the techniques described herein.

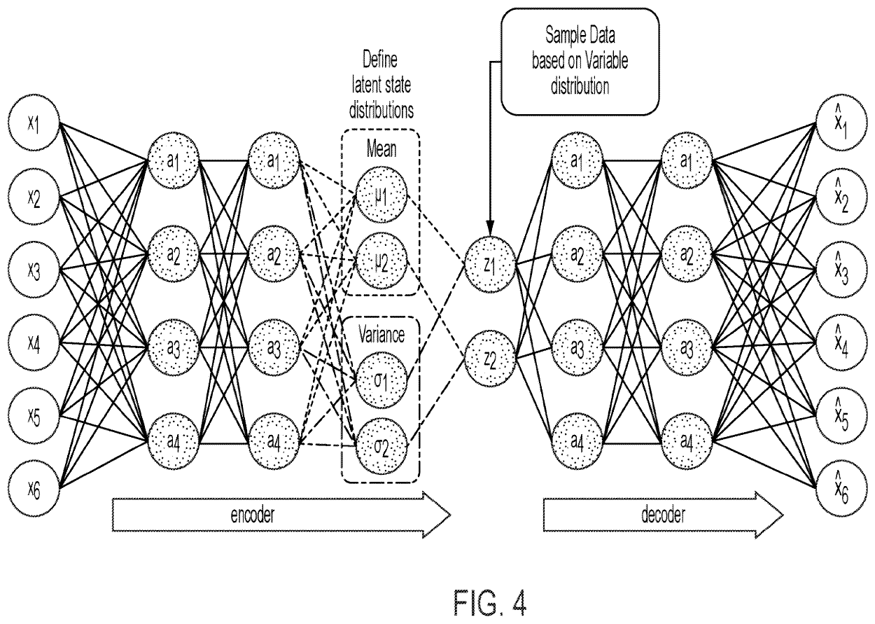

Process 200 then proceeds to act 212, where a variational autoencoder (VAE) is used to generate augmented data for people within each of the plurality of stress level categories based on the data received in act 210. Autoencoders consist of two networks, an encoder, and a decoder. The encoder maps high-dimensional input data into a latent space. The decoder uses the latent space to reconstruct input data as output. VAEs provide a probabilistic way to describe the data in latent space by varying the architecture of an autoencoder. For example, rather than providing a single value as output as in a typical autoencoder architecture, a VAE provides a range of output values based on a statistical distribution. Typical VAEs select a Gaussian distribution for the encoding process. VAEs also include a decoder configured receive a sample vector of features from the distributions of the features in the latent space, and generate an output that is similar to, but slightly different from, the feature set provided as input to the VAE. In this way, VAEs are sometimes referred to as generative models that can be used to generate synthetic data. An example of a VAE architecture is shown in FIG. 4.

The inventors have recognized and appreciated that conventional VAEs that encode and sample data using Gaussian distributions may not work well to generate augmented data having different data types, which may be used to train a predictive model of stress. Accordingly, some embodiments of the technology described herein use different distributions for encoding and sampling the data into and from latent space based on the type of feature input to the VAE. Continuing with the example above, the collected training data may include three types of data (categorical data, binary data, and continuous data) for users having three different stress levels (low, moderate, and high stress). The training data may be segregated into subsets based on the type of input data and stress level, and each subset may be processed using the VAE configured to use a distribution that corresponds to the particular data type being processed. For instance, in a first pass, categorical data for low stress people may be provided as input to the VAE configured to use a Poisson distribution for encoding the data into the latent space and sampling the data from latent space. The resulting output of the first pass of the VAE may be augmented data for low stress people that includes a set of values for categorical data. In a second pass, binary data for low stress people may be provided as input to the VAE configured to use a Bernoulli distribution for encoding the data into the latent space and sampling the data from latent space. The resulting output of the second pass of the VAE may be augmented data for low stress people that includes a set of values for binary data. In a third pass, continuous data for low stress people may be provided as input to the VAE configured to use a Gaussian distribution for encoding the data into the latent space and sampling the data from latent space. The resulting output of the third pass of the VAE may be augmented data for low stress people that includes a set of values for continuous data. The output of the first, second and third passes can then be combined to provide augmented data for low stress people that includes multiple different data types (e.g., categorical, binary, continuous).

A similar process may be performed for the training data for other stress levels (e.g., moderate and high stress), resulting in a total of nine passes through the VAE to generate augmented data for individuals within each of the three stress levels. Although processing the initial training data set with a VAE is described herein as operating in a serial manner (e.g., by using nine serial passes), it should be appreciated that at least some of the processing may occur in parallel using any suitable number of VAEs, and aspects of the disclosure not limited in this respect. After generating the augmented data for different stress level individuals, process 200 proceeds to act 214, where the augmented data is output as an augmented training data set that may be used to train a predictive model of travel-related stress.

In initial testing of the architecture it was shown that the generated augmented data was similar to the original data used to generate the augmented data, confirmed using a z test, and inclusion of the augmented data as training data resulted in significantly improved model accuracy for predicting travel-related stress compared to when only collected training data and not augmented data was used to train the model.

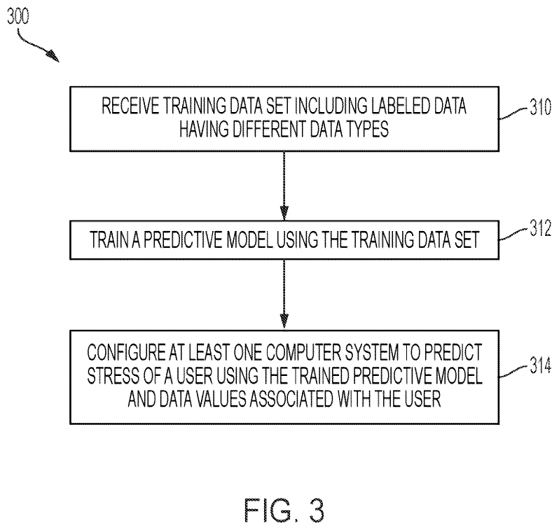

FIG. 3 illustrates a process 300 for training a predictive model using labeled data in accordance with some embodiments of the present disclosure. In act 310, a training data set including labeled data having different data types is received. For instance, in the example described in connection with process 200 of FIG. 2, augmented data is generated that includes three different data types categorical data, binary data and continuous data. Each of the instances of the augmented data is associated with a label indicating a stress level (e.g., low, moderate, high stress) associated with the feature values in that instance. The augmented data generated in process 200 is one example of a training data set including labeled data that may be received in act 310 of process 300. Process 300 then proceeds to act 312, where a predictive model may be trained using the training data set. In some embodiments, the predictive model may be implemented, at least in part, as a neural network, and training the predictive model may entail determining a set of weights for nodes of the neural network that enable the neural network to discriminate users having different stress levels.

Process 300 then proceeds to act 314, where at least one computer system is configured to predict stress of a user using the trained predictive model and data values associated with the user. For instance, a mobile computing device of the user may have installed thereon an app for a travel-related web-based application. The mobile computing device may be configured to record data values (e.g., typing speed, travel speed, etc.) associated with the user, and the data values may be provided by the app on the user's mobile computing device to the travel-related web-based application hosted on a server or other computer system via a network. The web-based application may receive the data values and provide them as input to a trained predictive model for predicting the user's current stress level (e.g., low, medium, high stress).

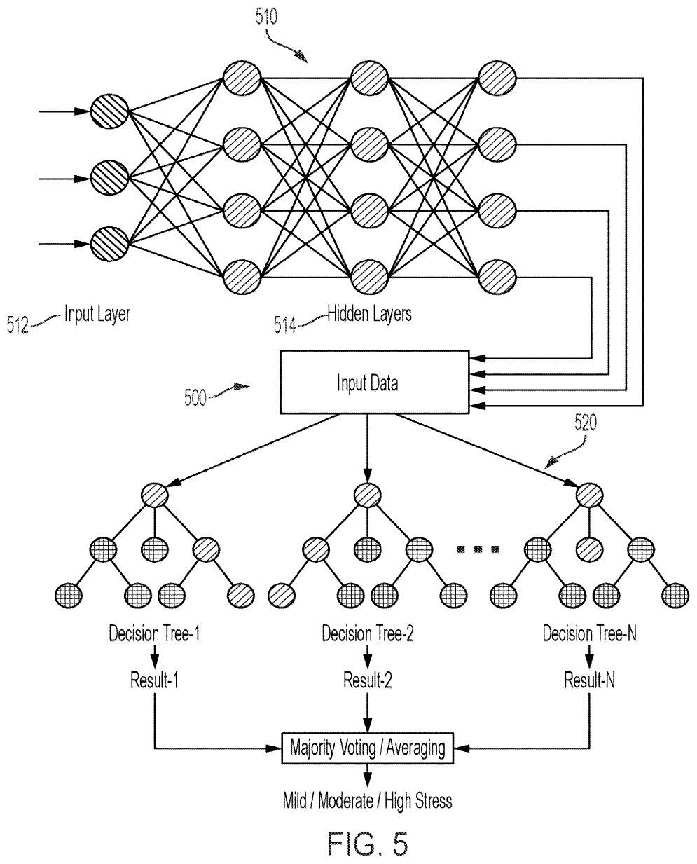

FIG. 5 schematically illustrates an example predictive model architecture 500 in accordance with some embodiments of the present disclosure. Architecture 500 includes an artificial neural network (ANN) 510 having output coupled to a decision tree network 520. ANN 510 may be implemented as a multi-layered perceptron including an input layer 512 of nodes, one or more hidden layers 514 of nodes, and an activation function that is used to determine the output of the ANN 510. As shown in FIG. 5, the output of the ANN 510 may be provided as an input to decision tree network 520. In some embodiments, decision tree network 520 may be implemented as a random forest (RF) model. The output of the decision tree network 520 may be a classification of the user as belonging to one of a plurality of stress groups (e.g., low, mild, medium, high stress).

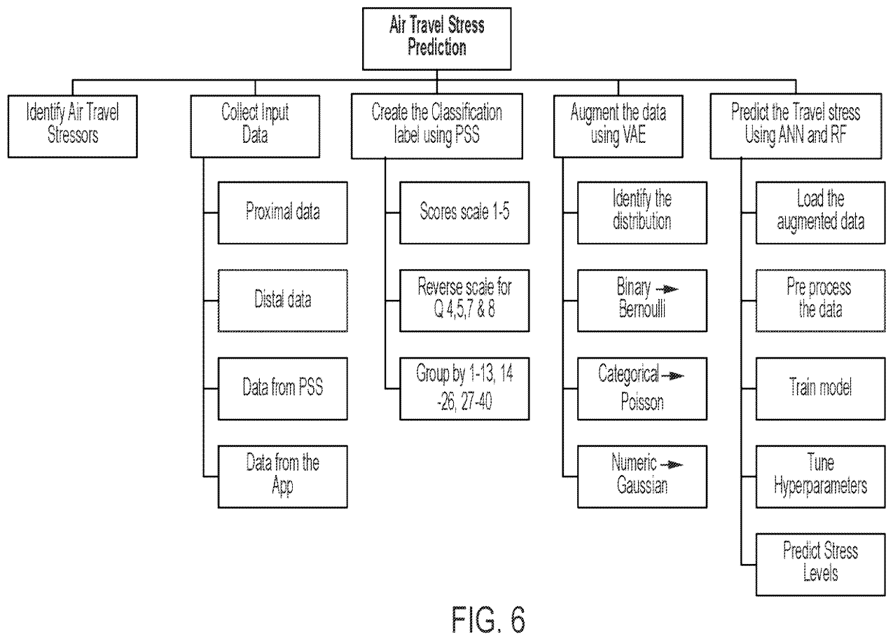

FIG. 6 schematically illustrates an overall process for predicting travel stress in accordance with some embodiments of the present disclosure. As shown, data collected from a plurality of different sources (e.g., proximal data, distal data, data from questions associated with a PSS score, data from a travel-related application, etc.) may be collected. Particular users associated with the collected data may be classified (e.g., using a PSS score) to create training data for training a model for predicting travel stress. Augmented data may then be generated using a modified VAE architecture, an example of which is described herein. After training the predictive model with the training data and the augmented data, the trained predictive model, an example architecture of which is shown in FIG. 5, may be used to predict stress levels of travelers.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above, and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification, unless clearly indicated to the contrary, should be understood to mean “at least one.”

The phrase “and/or,” as used herein in the specification should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of and “consisting essentially of shall be closed or semi-closed transitional phrases, respectively.

1. A method, comprising:
receiving, for each person from a plurality of people, data associated with a stress level of that person, the data including data of a first data type and data of a second data type;
categorizing each person from the plurality of people as belonging to a stress group from a plurality of stress groups;
generating, using a variational autoencoder configured to use a first distribution to encode and sample data of the first data type and a second distribution to encode and sample data of the second data type, augmented data for each stress group from the plurality of stress groups, the augmented data including data having the first data type and data having the second data type; and
outputting the augmented data as training data for training a machine learning model to predict stress.

2. The method of claim 1, wherein the first data type is categorical and the second data type is continuous.

3. The method of claim 1, wherein the first distribution is a Poisson distribution and the second distribution is a Gaussian distribution.

4. The method of claim 1, wherein the data also includes a third data type, the variational autoencoder is further configured to use a third distribution to encode and sample data of the third data type, and the augmented data includes data having the third data type.

5. The method of claim 4, wherein the first data type is categorical, the second data type is continuous and the third data type is binary.

6. The method of claim 4, wherein the first distribution is a Poisson distribution, the second distribution is a Gaussian distribution, and the third distribution is a Bernoulli distribution.

7. The method of claim 1, wherein the plurality of stress groups includes a low stress group and a high stress group.

8. The method of claim 1, wherein the plurality of stress groups includes a low stress group, a moderate stress group, and a high stress group.

9. The method of claim 1, wherein generating the augmented data comprises:
providing, as a first input to the variational autoencoder, data of the first data type for a first stress group from the plurality of stress groups;
encoding, by an encoder of the variational autoencoder, features of the data of the first data type in a latent space according to the first distribution;
sampling first feature values for the data of the first data type encoded in the latent space;
decoding, by a decoder of the variational autoencoder, the sampled first feature values to generate first output data having the first data type;
providing, as a second input to the variational autoencoder, data of the second data type for the first stress group of the plurality of stress groups;
encoding, by the encoder of the variational autoencoder, features of the data of the second data type in the latent space according to the second distribution;
sampling second feature values for the data of the second data type encoded in the latent space;
decoding, by the decoder of the variational autoencoder, the sampled second feature values to generate second output data having the second data type; and
combining the first output data and the second output data to generate the augmented data for the first stress group.

10. A non-transitory, computer-readable medium storing instructions that, when executed by a processor, cause the processor to:
receive, for each person from a plurality of people, data associated with a stress level of that person, the data including data of a first data type and data of a second data type;
categorize each person from the plurality of people as belonging to a stress group from a plurality of stress groups;
generate, using a variational autoencoder configured to use a first distribution to encode and sample data of the first data type and a second distribution to encode and sample data of the second data type, augmented data for each stress group from the plurality of stress groups, the augmented data including data having the first data type and data having the second data type; and
cause an output of the augmented data as training data for training a machine learning model to predict stress.

11. The non-transitory, computer-readable medium of claim 10, wherein the first data type is categorical and the second data type is continuous.

12. The non-transitory, computer-readable medium of claim 10, wherein the first distribution is a Poisson distribution and the second distribution is a Gaussian distribution.

13. The non-transitory, computer-readable medium of claim 10, wherein the first data type is categorical, the second data type is continuous, the first distribution is a Poisson distribution and the second distribution is a Gaussian distribution.

14. The non-transitory, computer-readable medium of claim 10, wherein the data also includes a third data type, the variational autoencoder is further configured to use a third distribution to encode and sample data of the third data type, and the augmented data includes data having the third data type.

15. The non-transitory, computer-readable medium of claim 14, wherein the first data type is categorical, the second data type is continuous and the third data type is binary.

16. The non-transitory, computer-readable medium of claim 14, wherein the first distribution is a Poisson distribution, the second distribution is a Gaussian distribution, and the third distribution is a Bernoulli distribution.

17. The non-transitory, computer-readable medium of claim 10, wherein the plurality of stress groups includes a low stress group, a moderate stress group, and a high stress group.

18. The non-transitory, computer-readable medium of claim 10, wherein the first distribution is a Poisson distribution, the second distribution is a Gaussian distribution, and the plurality of stress groups includes a low stress group, a moderate stress group, and a high stress group.

19. The non-transitory, computer-readable medium of claim 10, wherein the plurality of stress groups includes a low stress group and a high stress group.

20. The non-transitory, computer-readable medium of claim 10, wherein generating the augmented data comprises:
providing, as a first input to the variational autoencoder, data of the first data type for a first stress group from the plurality of stress groups;
encoding, by an encoder of the variational autoencoder, features of the data of the first data type in a latent space according to the first distribution;
sampling first feature values for the data of the first data type encoded in the latent space;
decoding, by a decoder of the variational autoencoder, the sampled first feature values to generate first output data having the first data type;
providing, as a second input to the variational autoencoder, data of the second data type for the first stress group of the plurality of stress groups;
encoding, by the encoder of the variational autoencoder, features of the data of the second data type in the latent space according to the second distribution;
sampling second feature values for the data of the second data type encoded in the latent space;
decoding, by the decoder of the variational autoencoder, the sampled second feature values to generate second output data having the second data type; and
combining the first output data and the second output data to generate the augmented data for the first stress group.
